(19) United States
(12) Patent Application Publication
(10) Pub. No.: US 2014/0363526 A1
(43) Pub. Date: Dec. 11, 2014

(54) SYNERGISTIC FORMULATION OF PLANT EXTRACTS FOR HEPATIC AND ADRENAL DISORDERS

(71) Applicant: Bioved Pharmaceuticals, Inc., San Jose, CA (US)

(72) Inventors: DEEPA CHITRE, LOS GATOS, CA (US); NARENDRA BHATT, MUMBAI (IN); DEBENDRANATH DEY, FREMONT, CA (US); SUNETRA CHASKAR, PUNE (IN)

(73) Assignee: BIOVED PHARMACEUTICALS, INC., San Jose, CA (US)

(21) Appl. No.: 14/298,524

(22) Filed: Jun. 6, 2014

Related U.S. Application Data
(60) Provisional application No. 61/832,053, filed on Jun. 6, 2013.

(51) Int. Cl. A61K 36/48; A61K 36/185; A61K 36/19

(52) U.S. Cl. CPC A61K 36/48; A61K 36/19; A61K 36/185
USPC 424/725

(57) ABSTRACT

A composition of matter comprising a synergistic formulation of plant extracts for hepatic and adrenal disorders is provided. In an implementation, an example extraction method for concentrating pharmacologically active fractions of plants provides a synergistically active compound containing approximately 5%-25% extract of Andrographis paniculata, approximately 10%-30% extract of Boerhavia diffusa, approximately 25%-50% extract of Phyllanthus niruri, and approximately 15%-40% extract of Tephrosia purpurea. The synergistically active compound provides greater therapeutic benefit than the individual ingredients alone, or the combined ingredients in different concentration ranges. The synergistic formulation may also be used for treatment of other medical conditions.

### RELATED APPLICATIONS

This patent application claims the benefit of priority to U.S. Provisional Patent Application No. 61/832,053 to Dey et al., filed Jun. 6, 2013 and incorporated by reference herein in its entirety.

### BACKGROUND

The liver plays a vital role in many bodily functions including, but not limited to, glucose homeostasis, digestion, cleansing, detoxification, protein production, blood clotting; cholesterol, fat and iron metabolism. There is a tremendous surge of liver related morbidity and mortality in recent years. Drug and alcohol induced liver failure is one of the key problems in modern day society. A variety of illnesses can affect the liver apart from environmental factors. If untreated, in chronic liver disease the normal liver cells or hepatocytes are replaced by scar tissue, also called as cirrhosis with hardening and non-functionality of the organ. Symptoms of liver diseases include weakness and fatigue, weight loss, nausea, vomiting, discoloration of the conjunctivae (icterus), skin and nails (jaundice) and eventual death.

The Liver

The liver is the largest solid organ and the largest gland in the human body. Located in the upper right portion of the abdomen and protected by the rib cage, it has two main lobes that are made up of tiny lobules. The liver cells have two different sources of blood supply. The hepatic artery supplies oxygen rich blood that is pumped from the heart, while the portal vein supplies nutrients from the intestine and the spleen. This is the vein which carries all the fatty acids, glucose, and amino acids after break down in the digestive tract. This portal vein also carries any foreign materials, alcohols, medicines and drugs directly take to the liver as a ‘safety gate’ for detoxification or filtration.

The liver has an almost miraculous ability to biochemically transform, break down, store, eliminate, and build up the plethora of chemicals to which it is exposed. Besides the skin, the liver is the only other organ in the body that is able to regenerate itself. The liver is involved in thousands of biochemical mechanisms making it second only to the brain in importance and complexity. As part of its glandular function, the liver makes bile, a fluid that contains among other substances, water, chemicals, and bile acids (made from stored cholesterol in the liver). Bile is stored in the gallbladder and when food enters the duodenum (the first part of the small intestine), bile is immediately let into the duodenum for digestion of the food. The liver is responsible for many critical functions within the body and once diseased or injured; the loss of those functions can cause significant damage to the body. Liver disease, also referred to as hepatic disease, is a broad term that covers all the potential problems that cause the liver to fail to perform its designated functions. Usually, more than 75% or three quarters of liver tissue needs to be affected before decrease in function occurs. Environmental pollutants, chemicals including from food, medicinals and alcohol; viral and other infections, excessive fat intake, pesticides, hormones, and stress all have detrimental effects on the liver.

Functions of the Liver

The functions of the liver are diverse, complex, and absolutely necessary for human metabolism and survival. They are:

a. Cleansing and Restoration—As one of the primary organs of detoxification, the liver is exposed to a vast array of food substances, chemicals, drugs and pollutants, especially in our modern world. The liver is the primary filter and detoxifying organ in the body.

b. Fat Metabolism—The liver is the primary metabolizer of fats and cholesterol, so it is particularly important to keep these substances circulating freely. It is very important to mobilize lipids and remove excess fats from the liver.

c. Antioxidant Defense—In the process of its metabolic and detoxification functions, the liver generates and is exposed to many free radicals. Several antioxidant compounds protect the protein, lipid and DNA of the liver from free radicals at cell membrane and intracellular fluid level.

d. Antioxidant Production—The liver synthesizes the antioxidant, glutathione, and the antioxidant enzyme, glutathione peroxidase. These are perhaps the most important antioxidants made by the body. Glutathione peroxidase contains four selenium atoms. It catalyzes the oxidation of glutathione, present in all animal cells at high concentration, and maintains the integrity of red blood cells.

e. Energy Generation—The liver requires a tremendous amount of energy to perform its diverse metabolic functions, which include maintaining a normal blood glucose concentration, forming cholesterol and phospholipids, and synthesizing amino acids, plasma proteins and other important compounds. Glucose is converted to its storage form glycogen in the liver. When required or in periods of stress, the liver converts glycogen back to glucose and makes it readily available to the other organs and tissues. In addition, this vital organ converts vitamins into their active co-enzyme forms and breaks down and excretes drugs and hormones.

Botanicals for Supporting Healthy Liver Function

In modern medicine, the treatment of a particular liver disease usually depends on its specific cause or a general support of the functions and recovery of the liver cells. Many botanicals have been used historically for promoting liver health. Today, modern research is confirming some of these benefits while shedding light on their mechanisms of action. There are several studies, both preclinical and clinical, showing some effect of different herbs in protecting liver functions. However, they were never properly isolated or activity checked in different assays. The active phytochemical fraction that imparts hepato-protective activity has also been identified in many plants. The biggest problems with these are lack of or marginal standardization, time of harvest and the percentage of active ingredients varies significantly. Most importantly, the individual and cumulative effect of these plants in a liver cell model has not been adequately demonstrated.

Nonalcoholic Fatty Liver Disease (NAFLD)

It is the most frequent cause of abnormal liver function tests in the United States with an estimated prevalence of 14-20%. It is caused by triglyceride (TG) accumulation within the liver and is strongly associated with insulin resistance, type 2 diabetes mellitus (T2DM), and the metabolic syndrome. Accumulating evidence suggests that hepatic lipid accumulation causes hepatic insulin resistance. For example, increasing hepatic lipid stores in mice by over-expressing lipoprotein lipase in the liver and in rats by short term high fat feeding results in liver-specific fat accumulation and hepatic insulin resistance. Several strategies have been employed to reduce hepatic steatosis or fat accumulation in rodents. These include treatment with a mitochondrial uncoupling agent (2,4-dinitrophenol), antisense oligonucleotide inhibition of acetyl-coenzyme A carboxylase I and II, adenoviral overexpression of malonyl-CoA decarboxylase, and transgenic overexpression of uncoupling protein 1, all of which successfully ameliorated hepatic insulin resistance. High fat diet also induces TG accumulation in liver (fatty liver syndrome) in mice which also shows abnormal glucose tolerance. So in dexamethasone (Dex) and insulin stressed condition if the TG synthesis in pre-adipocytes can be blocked, that can be an advantage to see development of new compounds or herbal formulations for Cushing's syndrome or diabesity and its complications. In this work, it was shown that the synergistic effect of four plant extracts does a better job when they are extracted by a proprietary method not by regular hydro-alcoholic fractionation. These effects could be a synergistic effect on TG synthesis under long term glucocorticoid treatment. Fatty liver disease can lead to long term and serious cardiovascular disease.

Nonalcoholic Steatohepatitis (NASH)

Nonalcoholic steatohepatitis or NASH is the most extreme form of NAFLD and is considered as a major cause of cirrhosis of liver of unknown etiology. This is a common, often “silent” liver disease. It resembles alcoholic liver disease, but occurs in people who drink little or no alcohol. The major feature in NASH is fat in the liver, along with inflammation and damage. Most people with NASH feel well and are not aware that they have a liver problem. Nevertheless, NASH can be severe and can lead to cirrhosis, in which the liver is permanently damaged and scarred and no longer able to work properly. NASH affects 2 to 5 percent of Americans with an estimated 6 million having active disease, and 600,000 having NASH related cirrhosis. An additional 10 to 20 percent of Americans have fat in their liver, but no inflammation or liver damage, a condition called “fatty liver.” Although having fat in the liver is not normal, by itself it probably causes little harm or permanent damage. If fat is suspected based on blood test results or scans of the liver, this problem is called nonalcoholic fatty liver disease (NAFLD). If a liver biopsy is performed in this case, it will show that some people have NASH while others have simple fatty liver. Worldwide, the incidence of NASH in general population is 10-20% and 37% among obese individuals. NAFLD affects 20-40% of the world's general population and up to 75% of obese individuals. Both conditions are becoming more common, possibly because of the greater incidence of obesity.

NASH is usually a silent disease with few or no symptoms. Patients generally feel well in the early stages and only begin to have symptoms—such as fatigue, weight loss, and weakness—once the disease is more advanced or cirrhosis develops. NASH is usually first suspected in a person who is found to have elevations in liver tests that are included in routine blood test panels, such as alanine aminotransferase (ALT) or aspartate aminotransferase (AST). When further evaluation shows no apparent reason for liver disease (such as medications, viral hepatitis, or excessive use of alcohol) and when X rays or imaging studies of the liver show fat, NASH is suspected. The only means of diagnosis is a liver biopsy. The progression of NASH can take years, even decades. The process can stop and, in some cases, reverse on its own without specific therapy. Or it can slowly worsen, leading to scarring, hardening and unable to function normally. At this stage, few treatments can halt the progression; and manifests as fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

Although NASH has become more common, its underlying cause is still not clear. It most often occurs in persons who are middle-aged and overweight or obese. Several factors are possible candidates:

insulin resistance

release of toxic inflammatory proteins by fat cells (cytokines)

oxidative stress (deterioration of cells) inside liver cells

Conventional Treatment of NAFLD and NASH

Conventionally, no specific therapies for NAFLD and NASH exist. The most important recommendations given to persons with this disease are to:

reduce their weight (if obese or overweight)

follow a balanced and healthy diet

increase physical activity

avoid alcohol

avoid unnecessary medications

Experimental approaches under evaluation in patients with NASH include antioxidants, such as vitamin E, selenium, and betaine; which act by reducing the oxidative stress and the use of newer anti-diabetic medications such as metformin, rosiglitazone, and pioglitazone—even in persons without diabetes. The latter is to combat insulin resistance, which most patients with NASH have.

Glucocorticoid induced Free Fatty Acid and Triglyceride synthesis

Glucocorticoids (GC) are steroid hormones secreted from the adrenal glands, under the influence of the hypothalamic-pituitary-adrenal (HPA) axis. The main glucocorticoid in humans is cortisol, and is an integral component of the response to stress. In times of stress, more glucocorticoids are secreted and influence the regulation of blood pressure, salt and water balance, immune function and cellular energy metabolism. Clinically, glucocorticoids are widely used as immunosuppressants directly regulating transcription via the glucocorticoid receptor (GR). The enzyme 11-β-hydroxysteroid dehydrogenase type 1 (11βHSD1) plays a crucial role in determining intracellular (pre-receptor level) GC levels by regenerating active GCs (cortisol) from inactive metabolites (cortisone) and is highly expressed in visceral adipose tissue (VAT). In humans, the adipogenic-enhancing properties of GCs are most obvious in the truncal obesity of Cushing's syndrome as well as in patients on systemic immunosuppressive corticosteroid treatment. The same effect is seen in humans during periods of stress where the glucocorticoid synthesis is up-regulated, and causes accumulation of fat deposits mainly in middle section of the body. GC excess manifests as raised blood pressure, VAT expansion, impaired peripheral glucose uptake, hepatic insulin resistance, increased protein catabolism and elevated plasma glucose levels, which are all part of the condition known as Metabolic Syndrome. It also causes fat accumulation in the liver and other body organs, and can lead to cardiovascular disease.

Cushing's Syndrome

Chronic glucocorticoid excess is called Cushing's syndrome, and leads to morbidity and mortality through a variety of factors, including obesity, osteoporosis, hypertension, hyperglycemia, impaired response to infection and accumulation of triglycerides in ‘ectopic’ sites such as the liver. Cushing's syndrome most commonly affects adults aged 20 to 50. People who are obese and have type 2 diabetes, along with poorly controlled blood glucose—also called blood sugar—and high blood pressure, have an increased risk of developing the disorder. This is especially important because an alarming number of patients are placed on long term exogenous steroids for conditions such as asthma, rheumatoid arthritis, inflammatory bowel disease, skin rashes and others. And with an ever growing level of stress in the modern world, human beings are exposed to chronic high levels of endogenously secreted cortisol starting from a very young age. Moreover, in Cushing's syndrome, there is marked redistribution of body fat, with accumulation in the depots in the abdomen, the nape of the neck and cheeks, but wasting of fat in many subcutaneous adipose compartments.

Liver Disease in Depression and Wilson's Disease

Traditional Chinese Medicine claims “If you are depressed, clean out your liver.” However it is unknown whether liver disease in depression is a cause or effect of disease and/or treatment. In the genetic condition called Wilson's disease, certain organs such as the liver, brain and kidneys are overloaded with large deposits of copper. So hepatic and renal failure and neurologic symptoms such as depression, irritability and gait disturbances are seen in parallel. A natural supplement to support the liver may help the depression in such cases.

Alcoholic Liver Disease (ALD)

Quantitatively, the liver is the major organ involved in the metabolic disposal of ethanol. The cytosolic alcohol dehydrogenase, microsomal ethanol-oxidizing system and peroxisomal catalase metabolize ethanol to acetaldehyde, which is a reactive metabolite that can produce injury in a variety of ways. Acetaldehyde is further metabolized to acetate by acetaldehyde dehydrogenase localized in the mitochondria and abundantly found in the liver. The rate of acetaldehyde formation is also highest in the liver. Hence the liver poses an early target for alcohol induced injury. Non-oxidative pathways of ethanol metabolism include formation of ethyl esters of long chain fatty acids, which also affect mitochondrial function.

Several risk factors inclusive of genetic predilections and malnutrition contribute to the cascade of events leading to sequential dysfunction of cells. This is manifested by cell membrane damage, hyper-metabolic state in the hepatocytes most prominent in the peri-venular area of the hepatic lobule, oxidative injury, steatosis, triggering of immune response, precipitation of cytoskeletal elements and production of collagen. The cytokines, thus released can also induce apoptosis in the liver cells. With this understanding of the pathogenesis of alcoholic liver disease, and in view of the relentless progression of the disease, there is a need for development and clinical evaluation of potential candidates for hepatoprotection.

ALD is a major cause of morbidity and mortality worldwide. The World Health Organization Global Alcohol Report, May 2014 states that in 2012, there were about 3.3 million deaths that were attributable to alcohol consumption and related illnesses. Worldwide, it is a leading risk factor in males aged 15-59 years. Globally, in 2012, 7.6% of all male deaths were attributable to alcohol, compared to 4.0% of female deaths.

Conventional Treatment Options in ALD

The goals of treatment in alcoholic liver disease are to reverse nutritional deficiencies, reduce inflammation of liver tissue, and to prevent scar tissue or cirrhosis. Besides abstinence and nutritional support, corticosteroids are utilized to reduce inflammation and increase survival. Steroids have significant side effects and are not recommended if a patient has failing kidneys, gastrointestinal bleeding or an infection. About 40 percent of people do not respond to corticosteroids. If corticosteroids do not work, pentoxifylline may be recommended. However, some studies of pentoxifylline have shown some benefit, others have not. Interferon-alpha (IFN-α) has been used in the treatment of viral hepatitis as well as in ALD. No alternative medicine treatments have so far been found to cure alcoholic hepatitis. The leaves and seeds of the milk thistle plant are thought to control inflammation in the liver and their supplements are a popular alternative treatment among people with liver disease. But studies have not found a benefit for people with alcoholic liver disease who take milk thistle supplements. Milk thistle is generally safe, but can cause diarrhea and nausea, and can interfere with prescription medications, hence not considered safe for use. SAMe is a supplement that's thought to reduce liver inflammation and help the liver repair itself. The human body naturally makes SAMe. Some evidence suggests people with liver disease have a deficiency of SAMe. In theory, taking a SAMe supplement might restore levels of the substance in the liver. But there is insufficient evidence to recommend use of SAMe for alcohol-related liver disease. SAMe is generally safe when taken as a supplement. However, side effects may include gas, nausea, vomiting and diarrhea; and it can interfere with prescription medications, hence not considered safe for use in ALD. Further issues with costs and ready availability limit the use of these agents for management of alcoholic liver diseases. Transplantation is now an accepted treatment in alcoholics who have brought their alcoholism under control and who benefit from adequate social support, but organ availability is still the major limiting factor and should be expanded more aggressively. Finally, abstinence from excessive drinking is always indicated; it is difficult to achieve, but agents that oppose alcohol craving are becoming available and they should be used more extensively.

Several drugs are under investigation for use in ALD. These include carvedilol, infliximab, metadoxine, polyenylphosphatidylcholine, remaxol and polyunsaturated lecithin; all of which have potential significant side effects. Experimental herbs described in literature for ALD include Hovenia dulcis, Cardiotopic pills, Danshen (Salviae miltiorrhizae), Panax notoginseng and Dryobalanops aromatica gaertn, CH-100, Osthole (Cnidium monnieri), caffeic acid, hexacholefit, Liv52, Colchicine, chrysin, tea polyphenol, Asparagus racemosus Willd. Root extracts, Kudzu, Ginkgo biloba, Curcumin, aged black garlic, Tamarix nilotica, Justicia schimperiana (Hochst. ex Nees) (Acanthaceae), Codonopsis lanceolata and Verbascum sinaiticum Benth, cinnamon, Beta vulgaris L., Cyperus alternifolius, Fenugreek (Trigonella foenum graecum), wild basil (Ocimum gratissimum), Puerariae radix, Paeonia moutan, Jianpi Huoxue and CH-100. However most of these are not validated or poorly demonstrated for their efficacy in ALD and other liver disorders.

On this background, thus, there is a definite paucity of effective, safe and affordable therapies for prevention and management of alcoholic liver diseases. As conventional medicine pursues a more integrated approach to managing disease, select herbs that influence liver function are being revisited and evaluated for their overall health promoting effects. In addition, the herbal treasure chest of Ayurveda offers a host of new phyto-pharmaceutical products that can be used to manage a spectrum of liver related imbalances.

Drug Induced Hepato-Toxicity

Drug-induced hepatitis or toxicity is inflammation of the liver on ingestion of certain Over The Counter or prescription medicines. Commonly taken pain killers such as acetaminophen can cause liver toxicity in doses that are not significantly higher than therapeutic doses. Other Non-Steroidal Anti-Inflammatory Drugs such as Ibuprofen and Naproxen may also cause hepatitis. Prescription drugs such as anabolic steroids, birth control pills, anti-tuberculosis drugs, statins, common antibiotics such as the macrolides and tetracycline, and cancer chemotherapy agents are all metabolized by the liver; and are notorious for causing liver toxic effects. Similarly, in the elderly, there is potential of severe hepatic-toxicity by concomitant use of alcohol and certain prescription medicines. Since OTC and prescription treatment is limited for liver disease, the most common therapy is to stop the drug or reduce the dose significantly. However that may not always be possible, such as in tuberculosis, cardio-vascular conditions and cancer treatments. In such cases, a natural plant-based supplement may be the best alternative to safe and effective concurrent or complementary treatment.

Ayurvedic Basis of Treatment of Liver Disorders

Traditional systems of medicine like Ayurveda, Unani (Islamic medicine), Traditional Chinese Medicine and others have recognized liver as a physiologically important organ that is responsible for a variety of functions. According to Ayurvedic texts, the liver (Yakrut) is origin of raktavahasrotas i.e. part of the circulatory system. Liver diseases like Kamala (jaundice) and its types are explained in ancient Ayurvedic texts. At the same time, consumption of excessive alcohol and its effects which are similar to alcoholic liver diseases in modern medicine, such as loss of appetite, nausea, vomiting, malaise, etc. are described under the term ‘Madyatyay’. Its diagnosis and management is also mentioned. Similar descriptions are found for metabolic disorders with details on ‘cleansing’ the liver. Traditionally trained practitioners of Ayurvedic medicine recognize that balancing liver function is pivotal to ensuring overall health. In dealing with problems of the liver, the primary goal in Ayurveda is to enhance liver detoxification processes and help protect against further damage. Based on traditional use, the herbs are selected and combined for their ability to promote “balance” within the body and to nourish the liver and related functions, including digestion and bile acid secretion. Sometimes herbo-mineral preparations are used for optimal efficacy.

More than 100 plants are used in hundreds of formulations to treat a variety of liver related disorders. These herbs act on multiple biochemical pathways to nourish the body as a whole and support various organ systems especially the liver. In addition, the herbal treasure chest of ancient Ayurveda offers a host of new phytochemicals that can be used both preventatively and clinically to manage a spectrum of liver related imbalances. Conventional biomedicine offers novel pathways to understand and provide scientific support for use of such natural products, with novel applicability of these ingredients for wider universal use.

### BRIEF DESCRIPTION OF THE DRAWINGS

FIGS. 1-2 are diagrams of example body weight data among male and female rats in alcohol-treated and carbon tetrachloride-treated pharmacology model.

FIG. 3 is a diagram demonstrating an example effect of individual plants of the example compound on ethanol treated HepG2 cell line and shows that the relative cell viability by individual plants is less than in the example compound formulation.

FIG. 4 is a diagram showing an example dose dependent increase in ethanol treated cells in percent relative cell viability of the example compound compared with positive control of drugs.

FIG. 5 is a diagram showing cell cytotoxicity with the example compound, by cell titer blue method.

FIG. 6 is a diagram showing inhibition of dexamethasone-induced triglyceride synthesis.

FIG. 7 is a diagram showing laboratory results for double-blind placebo-controlled clinical study with the example compound.

### DETAILED DESCRIPTION

### Overview

In an implementation, an example compound combines multiple plant extracts (also referred to herein as “herbs” or “plant components”) for a synergistic therapeutic effect, and is developed and formulated according to Ayurvedic principles of treatment. The individual herbs in the example compound have been studied individually for their efficacy in various liver ailments. Example herbs were selected and combined for their ability to act on multiple biochemical pathways and promote “balance” within the human body, and to nourish the liver and its related functions, including digestion, bile acid secretion, detoxification, nourishment, protection, and immuno-modulation. In an implementation, the example compound consists of extracts of following four herbs:

1. Andrographis paniculata (Kalmegh)

2. Boerhavia diffusa (Punarnava)

3. Phyllanthus niruri (Bhuiamla)

4. Tephrosea purpurea (Sharpunkha)

Andrographis paniculata is conventionally used for a variety of ailments and has been shown to protect against toxin induced hepatotoxicity. Andrographis paniculata has been studied in various experimental models including trial scenarios of carbon-tetrachloride, galactosamine, and paracetamol-induced liver damage. The plant components have been observed to prevent lipid peroxidation, maintain balance of nitric oxide, endothelin and decrease effect of oxidative stress on the cells. The diterpenes in andrographis increase glutathione (GSH), which may decrease susceptibility of the tissue to oxidative damage.

Boerhaavia diffusa and Phyllanthus niruri are conventionally used for management of hepatic disorders and internal inflammations. Boerhaavia diffusa has been studied and found to have beneficial and significant effect on the liver function tests in terms of reduction and return to normal values.

Phyllanthus niruri has been studied extensively in management of viral hepatitis and also in carbon tetrachloride-induced and galactosamine-induced cytotoxicity in a model of primary cultured rat hepatocytes.

Tephrosia purpurea has been used in several hepatoprotective formulations and has been studied to have a stimulant effect on the drug-metabolizing enzymes when tested in combination with Andrographis paniculata and other herbs. The flavonoids in Tephrosia purpurea have been found to be effective inducers of Phase II enzymes and to modulate both the cell-mediated and the humoral components of the immune system. These activities should have a favorable effect in terms of early metabolism of alcohol to acetate and thus decrease the time of exposure of the hepatocytes to reactive metabolites like acetaldehyde. The flavonoids may also help in decreasing the immune mediated damage to the hepatocytes as already described.

Each of the individual herbs in the example compound is carefully selected to provide an overall combined and synergistic effect in liver disorders and especially in ALD and NASH. The extraction of each individual herb can be accomplished by a technique described in U.S. patent application Ser. No. 13/322,147 to Chitre et al, entitled “Method for Extraction of Fractions Containing Pharmacologically Active Ingredients with Less Cytotoxicity from One or More Plants” that yields one or more bioactive fractions that have higher therapeutic efficacy than extracts obtained by conventional methods.

In an implementation, the above-cited example method of Chitre et al extracts a pharmacologically active fraction from each corresponding herb by soaking the herb in, e.g., chloroform and aqueous 60% methanol for 8-12 hours, filtering the chloroform and aqueous 60% methanol to obtain a first residue and a first filtrate, settling the first filtrate to provide two immiscible layers, wherein the two immiscible layers are an aqueous methanol layer and a chloroform layer, separating the chloroform layer, concentrating the chloroform layer to obtain a first dry extract, dissolving the first dry extract in hexane, filtering the first dry extract dissolved in hexane to obtain a second residue and second filtrate, drying the second residue and storing as a first fraction, re-extracting the first fraction to obtain a second fraction, and combining the first and second fractions to provide the pharmacologically active fraction. The example method of Chitre et al may further include a de-pigmentation step, a de-fatting step, or a detoxification step to decrease a toxicity level of the pharmacologically active fraction. The de-pigmenting agent, de-fatting agent, or detoxifying agent may be pentane, hexane, heptane, diethyl ether, petroleum ether, ethylene chloride, methylene chloride, cyclohexane, or solvent ether, for example.

The example compound containing the four herbs described above has been tested in an alcohol-induced HepG2 cell model, against the efficacy of the individual plants alone in different doses, to prepare a synergistic formulation that therapeutically promotes liver health with more efficacy than the conventional herbs alone.

Example Studies and Experimentals

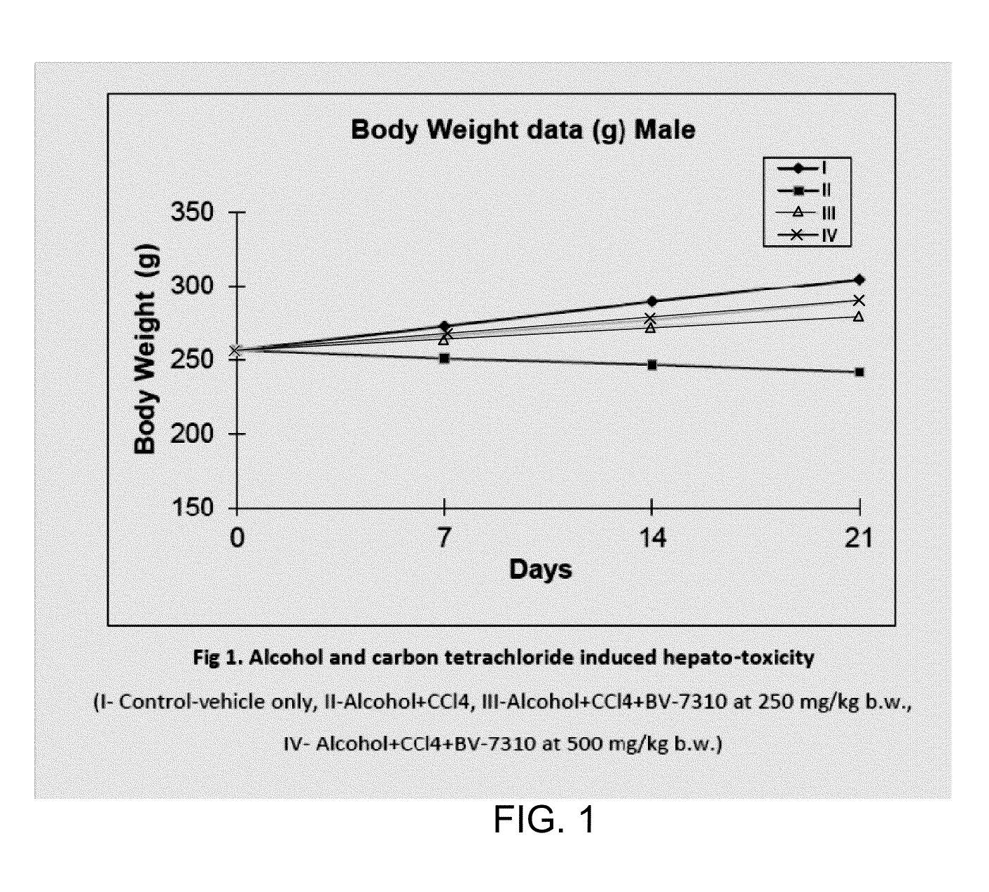

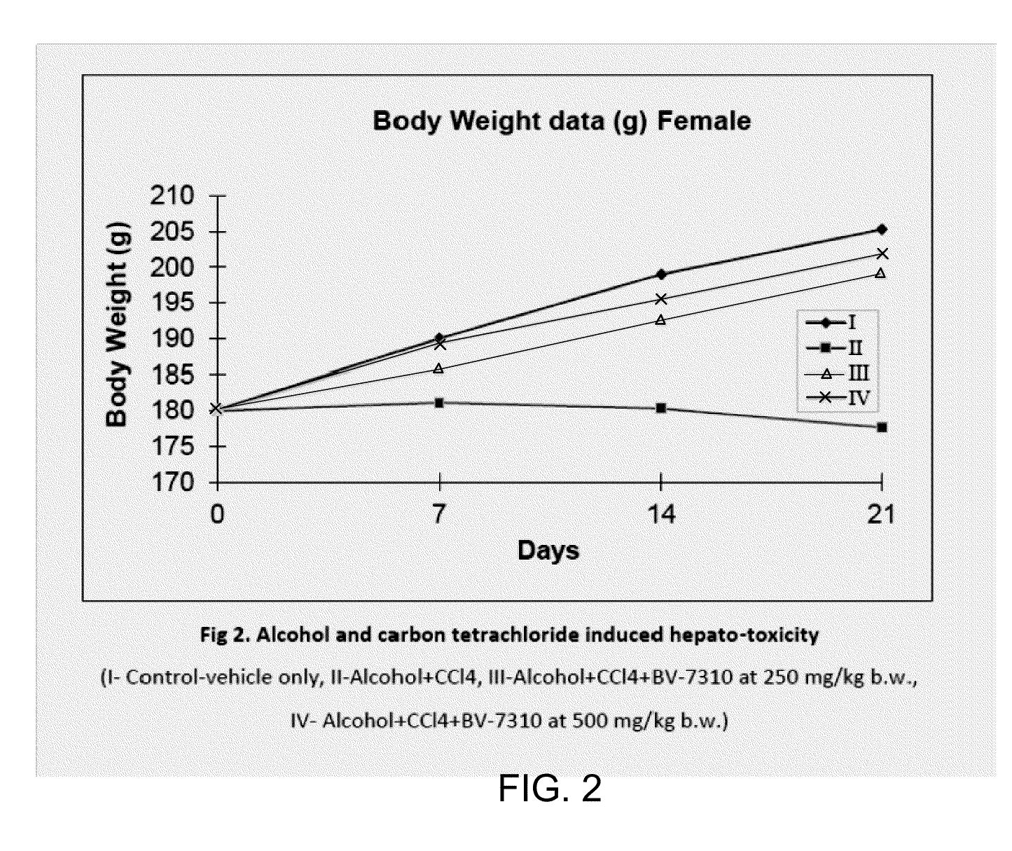

FIGS. 1-2 are diagrams of example body weight data among male and female rats in alcohol and carbon tetrachloride treated pharmacology model. Group I is control animals with no exogenous agent or drug. Group is animals treated with alcohol and carbon tetrachloride. Group III is animals treated with alcohol and carbon tetrachloride and an example compound in a dose of 250 mg/kg body weight. Group IV is animals treated with alcohol and carbon tetrachloride and the example compound in dose of 500 mg/kg body weight. Group IV and to some extent Group III shows body weight gain or maintenance comparable to that of control animals.

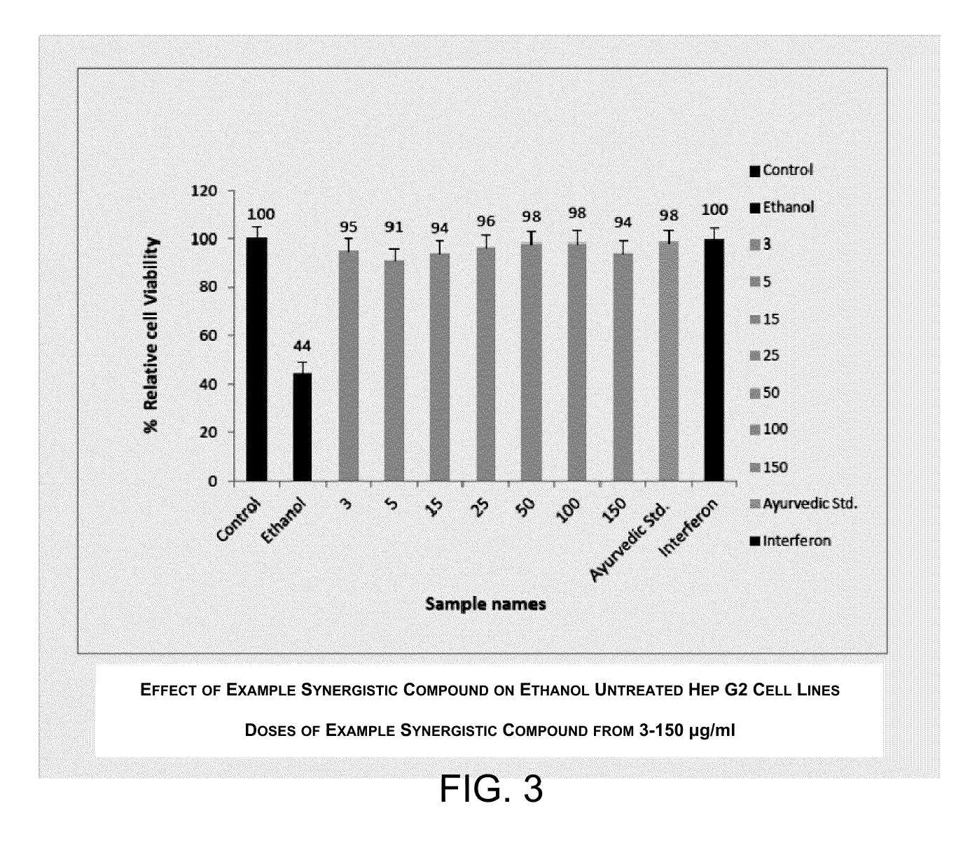

FIG. 3 demonstrates the effect of individual plants of the example compound on ethanol treated HepG2 cell line and shows that the relative cell viability by individual plants is less than in the example compound formulation.

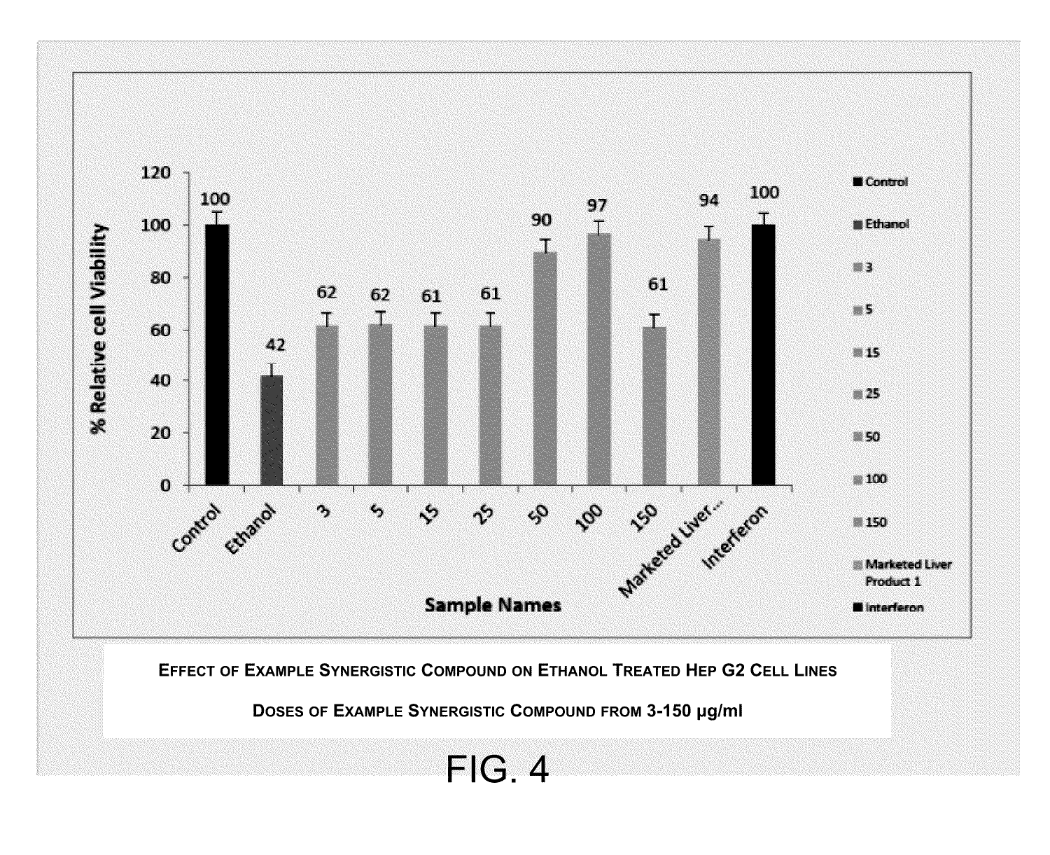

FIG. 4 shows the dose dependent increase in ethanol treated cells in percent relative cell viability of the example compound compared with positive control of drugs. The result was found to be statistically significant (P<0.05), when compared to ethanol treated cells alone (no study or control drug). The example compound drug concentration ranges from 3-100 μg/ml. Relative cell viability for 3-25 μg/ml concentration shows 61-62%, while 50 to 100 μg/ml shows 90 and 97% which was comparable with marketed popular Ayurvedic Drug and injectable prescription drug Interferon-α. Relative cell viability again decreases with increase in concentrations.

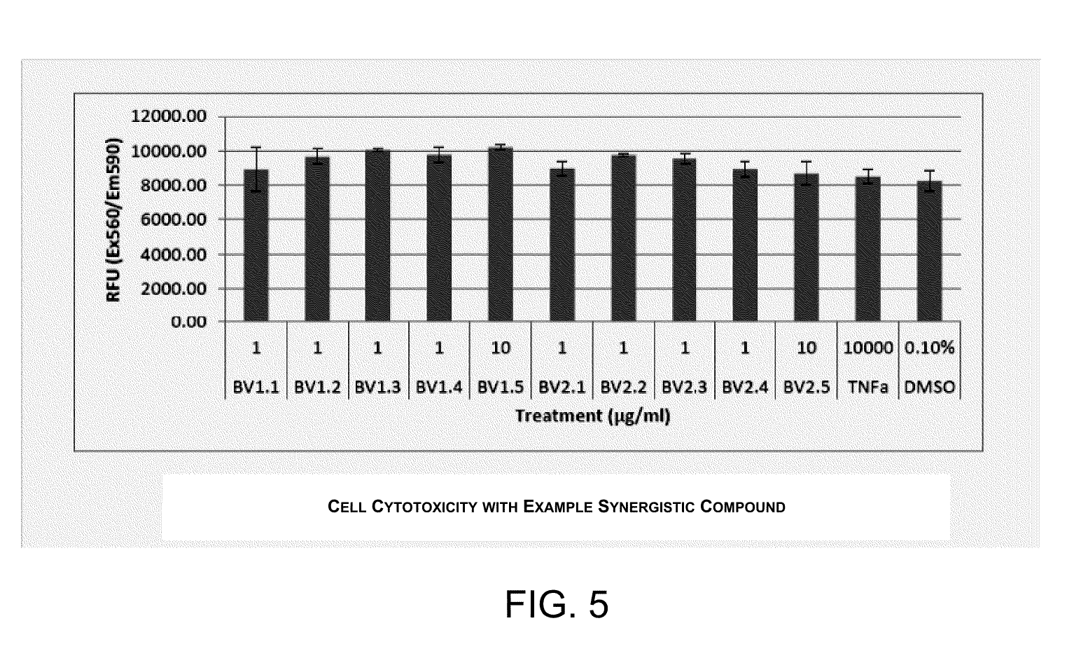

FIG. 5 shows cell cytotoxicity with the example compound, by cell titer blue method. Samples were coded as follows: BV 1.1-1.4 are individual four plant extracts using conventional methods of extraction, each at a dose of 1 μg/ml. BV 1.5—combination of 4 plant extracts using conventional methods. BV 2.1-2.4 are individual four plant extracts using novel patented methods of extraction, each at a dose of 1 μg/ml. BV 2.5—combination of 4 plant extracts using novel patented methods of extraction. No single plant extracts (at 1 μg/ml and formulation at 10 μg/ml) by extraction method BV 1.0 or BV 2.0 showed any toxicity to the human cells.

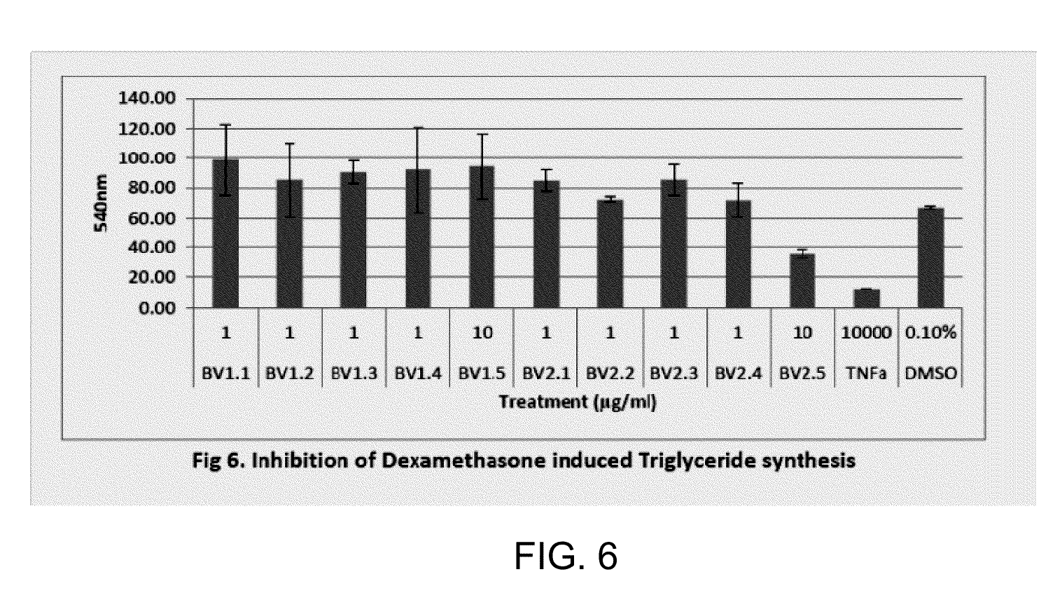

FIG. 6 shows inhibition of dexamethasone induced triglyceride synthesis. Plant extracts at 1 μg/ml or combined formulation at 10 μg/ml by an example extraction method did not inhibit any TG synthesis in these cells, whereas externally added TNF-a (a known inhibitor of this TG synthesis) significantly reduced TG synthesis compared to DMSO control. When a more proprietary extraction method, e.g., that of Chitre et al., above, was applied, all single plant extracts at 1 μg/ml did not significantly inhibit TG synthesis, but the mixed formulation BV 2.5 significantly reduced TG synthesis without any cytotoxic effects.

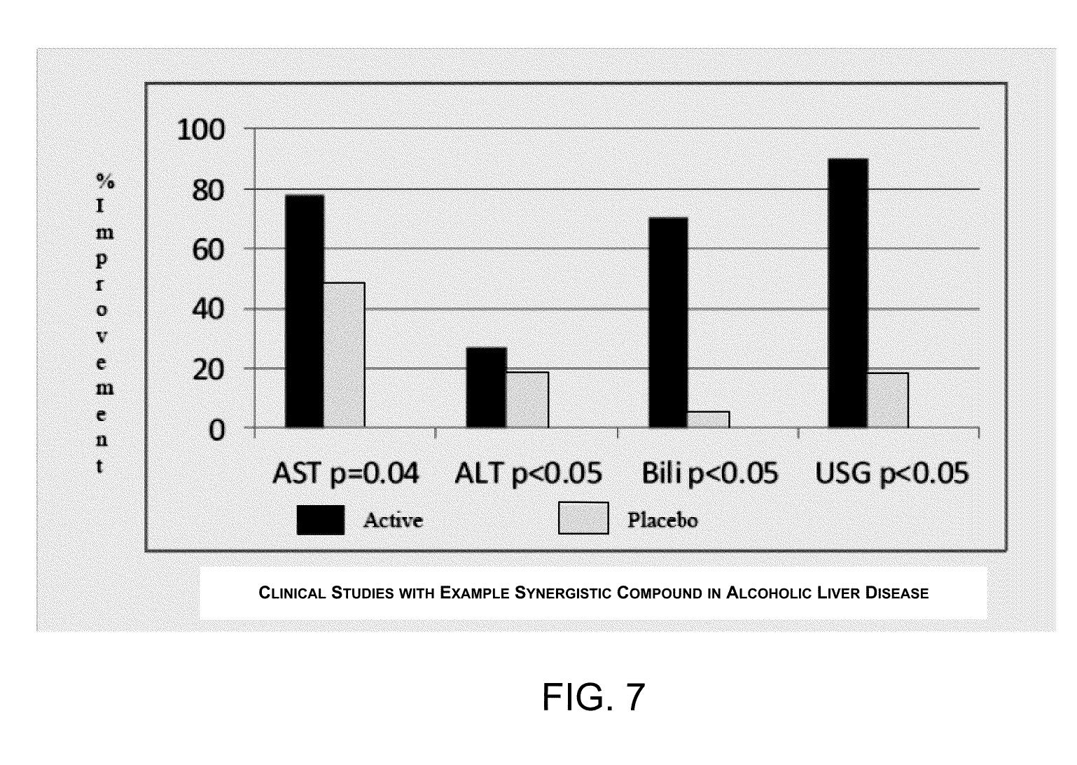

FIG. 7 shows laboratory results for double-blind placebo-controlled clinical study with the example compound, AST-Aspartate Aminotransferase, ALT-Alanine Aminotransferase, Bili-Bilirubin, USG-Ultrasound liver. The improvement in each of these parameters was statistically significant.

Example Chemistry of Individual Herb Extracts

1. Andrographis paniculata—The chemical constituents of A. paniculata include diterpene lactones viz., andrographolide, andrographanin, deoxyoxoandrographolide; glycosides viz., neoandrographolide, andrographiside and flavonols viz., oroxylin, wogonin, andrographidines A, B, C, D, E and F.

2. Boerhavia diffusa—The chemical constituents of B. diffusa include the glycoside punarnavoside, rotenoids viz., boeravinones A, B, C, D and E, lignans, flavones and sterols.

3. Phyllanthus niruri—The chemical constituents of P. niruri include lignans phyllanthin, hypophyllanthin, etc., and flavonoids such as quercetin, astralgin, rutin and other alkaloids.

4. Tephrosia purpurea—The chemical constituents of T. purpurea include flavonoids such as rutin, tephrosin, deguelin and quercetin.

Example Standardization

Standardization of the example compound, a polyherbal formulation containing the four plant extracts described above was achieved in phases.

Phase 1—Standardization of raw materials. The source of plant materials, identification, quality, and pharmacognostic characterization was done in collaboration with vendors at each vendor site.

Phase 2—Standardization of specialized extraction technology. This also was done in collaboration with the vendor. In an implementation, the example laboratory processes for extraction developed by the authors as described in U.S. patent application Ser. No. 13/322,147 to Chitre et al, entitled “Method for Extraction of Fractions Containing Pharmacologically Active Ingredients with Less Cytotoxicity from One or More Plants”, were scaled up by the vendor.

Phase 3—Development of TLC based identification of extracts and finished product for quantitative assay by HPLC method of each extract in the example compound's formulation.

Phase 4—The specifications were confirmed by analyzing several batches of extracts and finished products and by stringent stability testing so that the efficacy and safety of the finished product was reproducible.

The uniqueness of the example compound at first presented some challenges during standardization:

1. For confirmation of each extract, an elimination technique was used. Presence of the extract in granule solution was confirmed by noting the absence of similar peak pattern in the elimination sample.

2. A. paniculata spots cause overlapping to B. diffusa spots. The presence of B. diffusa was proven using the solution from which A. paniculata had been eliminated.

3. At very high concentrations (approx. 40 μl), P. niruri showed some interference with B. diffusa; but at normal concentrations used for testing, no such interference was observed.

The example compound's formulation was then tested systematically in a multitude of studies for safety and efficacy in liver conditions, especially in alcoholic liver disease. All the studies were conducted under the guidance and supervision of Institutional Ethics Committees and followed strict principles of OECD and ICH guidelines. The following are brief synopses of each of the studies and their results:

Pre-Clinical Studies

In-Vivo Studies

1. Acute Toxicity

Acute toxicity of the example compound was evaluated in the mouse model. Five male and five female Swiss albino mice were given a single dose of the example compound by oral gavage. the example compound at 5 g/kg body weight was administered as a suspension in distilled water. Mice were monitored daily for 14 days for body weight and for signs of toxicity and mortality. Treated mice exhibited a normal pattern of body weight gain over the study period with no mortality issues. On Day 15, necropsy was performed and specimens were subjected to gross pathological examination. The treated mice did not show any abnormal toxic or clinical signs. The median lethal dose (LD50) of the example compound by oral route in Swiss albino mice was determined to be more than 5 g/kg body weight.

2. Chronic Toxicity

The purpose of this study was to determine the toxicological profile of the example compound in the rat animal model. A total of 160 Sprague Dawley rats (80 males and 80 females) approximately 5-7 weeks old and weighing between 80-100 g each were randomly selected and allocated to treatment groups by weight; such that the mean body weights of each group were not significantly different. the example compound was administered orally at dose levels of 0.5, 1 and 2 g/kg body weight per day to rats for a period of 180 consecutive days. Additional group of rats were included for evaluation of reversibility of toxic effects; these were observed for 28 days post treatment. The animals were observed continuously for any adverse reaction, abnormal activity, motility, mortality etc. At the end of the experiment the animals were subjected to hematological, biochemical and histopathological studies. No abnormal changes in the hematological or biochemical parameters were observed. No abnormality was observed in the histopathological examination of the organs in the control and/or treated rats.

Summary

On the basis of overall findings of the toxicological studies, it was concluded that the example compound did not exhibit any toxic effects in rats after oral administration in doses of 500, 1000 and 2000 mg/kg body weight for the period of 180 days; or even at doses 5-10 times higher than the prescribed therapeutic dose.

3. Hepatoprotective Activity

The objective of the study was to test the effect of the example compound on Alcohol+Carbon-tetrachloride (CCl4) induced hepatotoxicity in rats. Healthy adult albino, Sprague Dawley rats (24 male and 24 female) weighing between 120 and 200 gm were divided into four groups. Group I rats were treated as control and received only the vehicle. Group II rats, serving as the positive control, received alcohol and CCl4. Group III and Group IV rats received alcohol, CCl4 and the example compound. Liver damage was induced by 40% ethanol (v/v, 2.0 ml/100 gm body weight P.O.) for 21 days. the example compound was administered orally to rats in Group III and IV for 21 days at a dose of 250 and 500 mg/kg respectively. Control animals received the vehicle alone at the same dosage volume for 21 days. All animals were dosed by oral gavage at approximately same time every day using a graduated syringe. On the 21st day, rats were bled under CO2 anesthesia and blood samples were withdrawn. Necropsy followed, with dissection of target organs to yield weight and volume of the liver and weight of the spleen. (FIGS. 1 and 2)

Throughout the experimental period, animals did not display any abnormal clinical signs. Group IV weight was comparable to Group I control during the test period. The dose 500 mg/kg appeared to stimulate appetite. Values of clotting time of male and female rats from Group I, II, III and IV were found to be comparable. Marked elevation of plasma levels of alanine aminotransferase, aspartate aminotransferase, alkaline phosphatase and bilirubin were observed in the positive control group (Group II) and a marked normalizing effect was observed in the example compound treated groups.

The absolute and relative weights of spleen in male rats from Groups II, III and IV were less when compared to Group I (control). The absolute and relative weights of liver and spleen of male rats from Group I, II and IV were comparable and the absolute and relative weights of liver and spleen of female rats in all groups were found to be comparable. The absolute and relative values of liver volume of male and female rats from Group II, III and IV were comparable to Group I

Microscopic examinations of liver tissues revealed acute congestion, round cell infiltration and degeneration with rats in Group II, III and IV displaying higher incidence than those in Group I. Group II and Group III rats had a higher incidence of slight acute congestion in liver and round cell infiltration was more prevalent in Group IV male rats. Focal degeneration was observed in four animals from Group II and in one animal from group III. Acute congestion and round cell infiltration in liver was also observed in few animals from Group I. Incidences of acute congestion and round cell infiltration were found to be higher in the positive control group than other groups. The overall findings of the study highlight the example compound (at both doses) displaying hepato-protective activity.

In-Vitro Testing

Hepatoprotective Effect of HepG 2 Cell Line

The formulation was further validated by in-vitro testing using human HepG2 cell lines (Human HepG2 carcinoma, ATCC 8065). The rationale was to show that the combination of the four active ingredients works synergistically better than any individual plant or component. Dose-dependent cytotoxicity of ethanol was found at higher concentrations (100-120 mM). Hence the hepato-toxic dose of ethanol was selected as 120 mM for 48 hr. The cells were then exposed to medium containing study formulation or medium alone and treated with toxicant ethanol respectively for 48 h. Additions of different concentrations of the example compound were used as test samples. A popularly marketed Ayurvedic herbal preparation with more than 15 different plants/herbs and Interferon-α were used as positive controls.

Results in FIG. 3 demonstrate the effect of Individual plants of Formulation the example compound on ethanol treated HepG2 cell line and shows that the relative cell viability by individual plants is less than in the combined the example compound formulation. Further, FIG. 4 shows the dose dependent increase in ethanol treated cells in percent relative cell viability of the example compound compared with positive control of drugs. The result was found to be statistically significant (P<0.05), when compared to ethanol treated cells alone (no study or control drug). the example compound drug concentration ranges from 3-100 g/ml. Relative cell viability for 3-25 μg/ml concentration shows 61-62%, while 50 and 100 μg/ml show 90 and 97% cell viability respectively. This was comparable with marketed popular Ayurvedic Drug and injectable prescription drug Interferon-α. Relative cell viability again decreases with increase in concentrations.

Inhibition of Long Term Dexamethasone Induced Triglyceride Synthesis in Human Subcutaneous Pre-Adipocytes

The study was conducted at a reputed US Contract Research Lab which has access to pooled human subcutaneous pre-adipocytes (female, average age 54.2, average BMI 27.9) from twelve different donors. These were cultured in 96-well plates in DMEM/Ham's F-12 (1:1, v/v) with 10% fetal bovine serum and differentiated according to a standard protocol. The next day, the pre-adipocyte medium was removed and replaced with differentiation medium; namely insulin, dexamethasone and IBMX (3-isobutyl-1-methylxanthine, a competitive nonselective phosphodiesterase inhibitor and a nonselective adenosine receptor antagonist. The media containing the formulations were prepared by different methods (individually and combined). After seven days, 90 μl of differentiation medium was removed and replaced with 120 μl of adipocyte medium (containing dexamethasone and insulin in DMEM). After seven more days, cells were assayed for cytotoxicity and cellular triglyceride content.

Cytotoxicity Assay by Cell Titer Blue Method

The cell titer blue kit was procured from Promega and followed the steps as per manufacturer's protocol. The assay is based on the ability of living cells to convert a redox dye (resazurin) into a fluorescent end product (resorufin). Non-viable cells rapidly lose metabolic capacity and thus do not generate a fluorescent signal. Medium was removed leaving 50 μl remaining in the wells. To each well, 10 μl of cell titer blue reagent was added and the cells were allowed to incubate for 2 hours at 37° C., 5% CO2. After 2 hours, 50 μl of conditioned medium was removed to a solid black bottom plate. The relative fluorescence of the conditioned medium was determined using the following wavelengths: 560 excitation/590 emission. The relative fluorescence units (RFU) were subtracted from the blank and test samples were compared to controls to determine cell cytotoxicity by measuring the live cells. No single plant extracts (at 1 μg/ml and formulation at 10 μg/ml) by extraction method BV 1.0 or BV 2.0 showed any toxicity to the human cells (FIG. 5)

Measurement of Cellular Triglycerides

Cellular triglycerides were measured by a two-step procedure. Triglycerides were first hydrolyzed by lipoprotein lipase to glycerol and free fatty acids by incubating Reagent A. Glycerol was then phosphorylated by adenosine-5′-triphosphate (ATP) forming glycerol-1-phosphate (G-1-P) and adenosine-5′-diphosphate (ADP) in the reaction catalyzed by glycerol kinase (GK). Next, G-1-P was oxidized by different steps using reagent B to a final end product, a quinoneimine dye that shows an absorbance maximum at 540 nm. Along with that a standard TG curve was done to derive the unknown values from the standard curve. After removing the conditioned medium for the cytotoxicity assay the cells were washed twice with PBS (phosphate buffered saline). After washing, all the PBS was removed and 15 μl of lysis buffer was added to the cells and incubated for 20 minutes at 37° C., 5% CO2. After 20 minutes, 135 μl of PBS was added to the wells followed by 20 μl of Reagent B. The cells were then incubated for 2 hours at 37° C., 5% CO2. After 2 hours the lysates were diluted in PBS in a fresh plate. An equal volume of Glycerol Reagent A was added to each well and allowed to incubate for 15 minutes at room temperature. After 15 minutes, the optical density of each well of the new plate was measured at 540 nm. The absorbance values of the unknown samples were converted to glycerol concentrations by correlation to the glycerol concentration standards using the generated linear regression equation. As shown in FIG. 6, plant extracts at 1 μg/ml or combined formulation at 10 μg/ml by regular extraction method (BV method 1.0 series) did not inhibit any TG synthesis in these cells, whereas externally added TNF-a (a known inhibitor of this TG synthesis significantly reduced TG synthesis compared to DMSO control. Interestingly when a more proprietary extraction method was applied (BV method 2.0 series), all single plant extracts at 1 μg/ml did not significantly inhibit TG synthesis, but the mixed formulation BV 2.5 significantly reduced TG synthesis without any cytotoxic effects. (FIG. 6)

The overall findings showed that the activity of the four plants in the example compound has a synergistic effect that was greater than the effect seen by any one of the plants individually. Further, this activity was greater than the popular marketed Ayurvedic plant based product and equivalent to that of Interferon-alpha.

Clinical Studies of the Example Compound in Alcoholic Liver Disease

A multicentric double-blind randomized placebo-controlled clinical study of 61 patients of alcoholic liver disease, was done over 12 weeks at two large teaching hospitals in Mumbai and Pune, India. 66 male patients aged 20-60 years, diagnosed with alcoholic hepatitis based on history of alcohol abuse (drinking more than 30 gm of alcohol since at least 5 years) and presence of clinical features and biochemical evidence (specified by AST or ALT>2 and increased Serum Gamma-Glutamyl Transferase (GGT), absence of serological markers of Hepatitis B (HbSAg) or C (anti HCV) were included in the study. Patients with cirrhosis of liver or decompensated liver disease, any severe/known systemic disease, use of any other investigational drug within 4 weeks of trial, use of corticosteroids, sylimarin or other therapies with hepato-protective or immunomodulator potential within 4-6 wks of trial were excluded from the study. Written informed consent was taken in all cases.

All selected patients received, in a randomized fashion, the example compound or placebo for 12 weeks. The patients were advised to stop alcohol but this was not mandatory. In case of patients continuing alcohol consumption despite advice, details of alcohol consumption during the trial was recorded wherever possible. The serum enzymes, other liver function tests and abdominal ultrasound were estimated at screening, at end of Week 1, 2, 4, 8 and 12. The hepato-protective activity of the example compound was assessed by comparison of change in serum enzymes between patients on the example compound and placebo. The safety parameters were tested at screening, and end of Week 4 and 12.

No abnormalities were obvious on general examination of patients on the example compound or placebo. The Hemoglobin count, RBC count, total and differential WBC count, platelet count, ESR, Blood sugar, Blood Urea Nitrogen, Serum creatinine and Urine routine and microscopic examinations did not show any significant changes over the course of treatment in either groups. These parameters are indicative of hematological abnormalities, renal dysfunction, chronic organic diseases, glycemic state and general health indices of the body.

Liver function tests, especially AST and ALT were considered the primary efficacy criteria. Serum GGT, total bilirubin, proteins, mean corpuscular volume of erythrocytes, alkaline phosphatase and prothrombin time were the other efficacy criteria. This was done considering that all patients with cirrhosis of liver were excluded from the study.

Sixty of the sixty-one patients studied showed increase in AST values though not greater than 300. An increase in the transaminases has been seen in 80-90% of patients with alcoholic liver disease. ALT was normal in fifteen of thirty-one patients on drug the example compound at initiation of therapy and in twenty-seven patients at end of treatment. Similar low ALT values have been observed in most patients with alcoholic liver disease. This discrepancy is seen because of pyridoxal 5′-phosphate depletion in alcohol abusers.

The improvement i.e. reduction in the AST value is significantly greater in the patients on the trial drug, the example compound as compared to placebo. Reduction in ALT values is also seen in the patients on the example compound though not to the extent seen with AST. The number of patients who have achieved normal values of ALT at end of study is higher with the example compound than the placebo. the example compound therefore helps in reduction and normalization of the transaminases, which indicates a role of the supplement in reduction of hepatocellular inflammation and necrosis.

Mean AST:ALT ratio at initiation of trial was greater than 2 in cases and controls. This ratio is highly suggestive of alcoholic liver disease. The change in ratio at end of trial is not statistically significant. And suggests that a study of longer duration may be warranted.

The GGT values at baseline were similarly increased in both patients on the example compound and placebo at initiation of therapy. GGT being an inducible enzyme is elevated in all forms of fatty liver, making it a sensitive indicator, though not specific, of liver disease. The improvement i.e. reduction in the GGT value is greater in the patients on the trial drug, the example compound as compared to placebo though not significant. GGT has a long half-life of 26 days, making its role limited in evaluation of liver disease. This could be a reason for not obtaining a significant difference in improvement though seen in the patients on drug, the example compound and those on placebo.

Serum alkaline phosphatase was not significantly affected by the course of therapy. Serum protein values showed marginal increase in patients on the example compound as compared to the group on placebo. Alkaline phosphatase is a marker of obstructive liver disease and therefore not a marker of alcoholic liver disease. Serum proteins are good markers of hepatic function in terms of synthesis. However, a rise in total proteins may be seen even in the presence of liver disease in case of an acute phase response, the reactants of which are synthesized in the liver.

Serum bilirubin was raised in twenty-three patients on the example compound, and in eighteen patients on placebo, at initiation of therapy. This was however not manifested, as icterus in some patients as the rise in bilirubin was less than 3 mg/dl in these instances. the example compound facilitated significant reduction in bilirubin values to normal values by the end of study. Hence, it can be assumed that the example compound helps restore the detoxification and excretory functions of liver which play a pivotal role in progression of alcoholic liver disease. Prolonged prothrombin time is an indicator of severe liver disease. The INR (International Normalized Ratio) has been devised as a standard means for avoiding inter-laboratory variations in the assessment of prothrombin time. There was no significant prolongation in either groups and reduction at the end of study was comparable in the two groups.

Maddrey's discriminant function, a fairly good indicator of prognosis in liver disease is derived from the prothrombin time and serum bilirubin. Discriminant function greater than 32 is considered a fairly good indicator of mortality and therefore bad prognosis in a patient of alcoholic liver disease. This index offers the advantage of few variables and easy computation, but is relatively imprecise. A higher reduction in number of patients with discriminant function above 32 is seen at end of four and eight weeks in the patients on the example compound. The ratio is reversed at the end of 12 weeks.

Most common findings on ultrasonography were bright echo texture of liver suggestive of fatty liver, free fluid in abdomen suggestive of ascites and hepatomegaly. The improvement on ultrasonographic examination of liver is highly significant in the patients on the example compound supplement. There was improvement in terms of reduction of size of liver, ascites and change in echo texture of the liver. (FIG. 7)

### CONCLUSION

Although only a few example embodiments have been described in detail above, those skilled in the art will readily appreciate that many modifications are possible in the example embodiments without materially departing from the subject matter. Accordingly, all such modifications are intended to be included within the scope of this disclosure as defined in the following claims

What is claimed is:

1. A composition of matter providing a synergistic therapy for alcoholic liver disease and nonalcoholic steatohepatitis, comprising:
approximately 5% to approximately 25% extract of Andrographis paniculata;
approximately 10% to approximately 30% extract of Boerhavia diffusa;
approximately 25% to approximately 50% extract of Phyllanthus niruri; and
approximately 15% to approximately 40% extract of Tephrosia purpurea.

2. The composition of matter of claim 1, wherein the synergistic therapy includes reducing inflammation of liver tissue and preventing cirrhosis of the liver.

3. The composition of matter of claim 1, wherein the synergistic therapy includes preventing and reducing fat formation in the liver, inflammation in the liver, and scar tissue damage in the liver.

4. The composition of matter of claim 1 further comprising at least one pharmaceutically acceptable excipient.

5. The composition of matter of claim 1, wherein the composition of matter is formulated as one of a tablet, a capsule, a powder, a suspension, and a solution.

6. The composition of matter of claim 1, wherein the composition of matter is effective in improving at least one of digestion, body metabolism, liver functions, bile secretion, detoxification and excretion capabilities of liver, immunomodulation, diuresis, and protection of liver from drug toxicity.

7. The composition of matter of claim 1, wherein the composition of matter is effective in lowering elevated plasma levels of at least one of alanine aminotransferase (ALT), aspartate aminotransferase (AST), alkaline phosphatase (ALP), in a mammal.

8. A synergistic composition, the synergistic composition comprising pharmacologically effective amounts of extracts obtained from plants of Andrographis paniculata, Boerhavia diffusa, Phyllanthus niruri, and Tephrosia purpurea.

9. The synergistic composition of claim 8, wherein the extracts are obtained from whole plant of Boerhavia diffusa, whole plant of Tephrosia purpurea, at least one of roots and leaves of Andrographis paniculata, and at least one of leaves and stem of Phyllanthus niruri.

10. The synergistic composition of claim 8, further comprising:
approximately 5% to approximately 25% extract of Andrographis paniculata;
approximately 10% to approximately 30% extract of Boerhavia diffusa;
approximately 25% to approximately 50% extract of Phyllanthus niruri; and
approximately 15% to approximately 40% extract of Tephrosia purpurea.

11. The synergistic composition of claim 8, wherein the synergistic composition is effective in ameliorating body ailments, the body ailments comprise at least one of loss of appetite, indigestion, alcoholic hepatitis, nonalcoholic steatohepatitis, fatty liver, alcoholic fatty liver disease, nonalcoholic fatty liver disease, Cushing's syndrome, metabolic syndrome, stress related liver and metabolic disorders, elevated serum bilirubin levels, drug induced liver toxicity, liver toxicity, ascites, hepatomegaly, hepatocellular inflammation and necrosis of hepatocytes.

12. The synergistic composition of claim 8, wherein the synergistic composition is effective in improving at least one of digestion, body metabolism, liver functions, bile secretion, detoxification and excretion capabilities of liver, immunomodulation, diuresis, and protection of liver from drug toxicity.

13. The synergistic composition of claim 8, wherein the synergistic composition is effective in lowering elevated plasma levels of at least one of alanine aminotransferase (ALT), aspartate aminotransferase (AST), alkaline phosphatase (ALP), in a mammal.

14. A method, comprising:
extracting a pharmacologically active fraction from each of the following herbs:
Andrographis paniculata;
Boerhavia diffusa;
Phyllanthus niruri;
Tephrosia purpurea;
wherein the extracting further comprises, for each herb:
soaking the herb in chloroform and aqueous 60% methanol for 8-12 hours;
filtering the chloroform and aqueous 60% methanol to obtain a first residue and a first filtrate;
settling the first filtrate to provide two immiscible layers, wherein the two immiscible layers are an aqueous methanol layer and a chloroform layer;
separating the chloroform layer;
concentrating the chloroform layer to obtain a first dry extract;
dissolving the first dry extract in hexane;
filtering the first dry extract dissolved in hexane to obtain a second residue and second filtrate;
drying the second residue and storing as a first fraction;
re-extracting the first fraction to obtain a second fraction, and combining the first and second fractions to provide the pharmacologically active fraction of the corresponding herb; and
after extracting each herb, then combining the herbs to compose a synergistically active compound containing:
approximately 5% to approximately 25% extract of Andrographis paniculata;
approximately 10% to approximately 30% extract of Boerhavia diffusa;
approximately 25% to approximately 50% extract of Phyllanthus niruri; and
approximately 15% to approximately 40% extract of Tephrosia purpurea.

15. The method of claim 14, further comprising administering the synergistically active compound to a person having one of an alcoholic liver disease condition or a nonalcoholic steatohepatitis condition.

16. The method of claim 14, further comprising administering the synergistically active compound to a person having one of a loss of appetite, an indigestion, an alcoholic hepatitis, a fatty liver condition, an alcoholic fatty liver, an elevated serum bilirubin level, a drug-induced liver toxicity, a liver toxicity, an ascites condition, a hepatomegaly condition, a hepatocellular inflammation, or a necrosis of hepatocytes.
